(19) United States
(12) Patent Application Publication
(10) Pub. No.: US 2025/0057624 A1
(43) Pub. Date: Feb. 20, 2025

(54) STERILE BARRIER PACKAGING SYSTEMS

(71) Applicant: Medtronic, Inc., Minneapolis, MN (US)

(72) Inventors: David Clarke, Loughrea (IE); Niall F. Duffy, Galway (IE); Bartosz R. Chamiga, Ennis (IE)

(21) Appl. No.: 18/723,338

(22) PCT Filed: Feb. 6, 2023
(86) PCT No.: PCT/IB2023/051040

§ 371 Date: Jun. 21, 2024

Related U.S. Application Data
(60) Provisional application No. 63/307,286, filed on Feb. 7, 2022.

(51) Int. Cl. A61B 50/30

(52) U.S. Cl. CPC A61B 50/30

(57) ABSTRACT

A sterile barrier system for medical device packaging. The sterile barrier system includes a layer forming a breathable sterile barrier. The layer includes a plurality of fibers. Each of the fibers includes a hollow fiber body and an agent contained within the hollow fiber body. The fibers are configured such that the agent flows from the corresponding hollow fiber body when the hollow fiber body is severed. When the sterile barrier system is subjected to damage, the agent is released from any hollow fiber body severed by the damage and imparts one or both of repair or visual indication of a possible integrity breach. In some embodiments, the agent is a self-healing agent. In other embodiments, the agent is a dye. In some embodiments, the layer is an electrospun web. A medical device packaged in a sterile barrier system is also provided.

### FIELD

The present disclosure generally relates to sterile barrier systems. More particularly, it relates to breathable sterile barrier systems useful, for example, as packaging for medical devices and medical devices packaged in sterile barrier systems.

### BACKGROUND

The use of sterile items in medical procedures is critical to the prevention of spreading harmful and infectious microbes to the patient. For this reason, items used in medical procedures are often provided to (and stored by) caregivers in packaging adapted to maintain sterility of the items prior to removal from the packaging. Packaging for medical devices, surgical products, and the like is expected to protect the contents of the packaging from physical damage and to act as a sterile barrier system to allow for sterilization of the contents of the package while acting as a microbial barrier to keep bacteria from contaminating the contents of the package.

The breathable sterile barrier utilized with medical item packaging is typically a flexible, film-like or web-like material. The breathable sterile barrier material can be secured over a plastic tray formatted to contain the medical item(s), can be secured onto itself in forming a bag-like structure, or other packaging formats. Regardless, the breathable sterile barrier material is desirably strong and durable. Well-known examples of breathable sterile barrier materials are available under the tradename Tyvek® from DuPont de Nemours, Inc. (e.g., Tyvek® 1073B, Tyvek® 1059B, and Tyvek® 2FS™). The Tyvek® material is a non-perforated, nonwoven product with microscopic pores, made of flashspun high-density polyethylene (>99%) fibers. Medical-grade papers are also sometimes used as a breathable sterile barrier packaging material.

### SUMMARY

The inventors of the present disclosure have recognized that existing breathable sterile barrier materials, while durable and generally resistant to damage, may nonetheless be ruptured or breached under various circumstances. When this occurs, it can be difficult for an end-user to visually perceive or detect existence or extent of a breach, for example due to the material structure and the size of the minimum damage that can otherwise impact an ability of the barrier to maintain sterility.

Some aspects of the present disclosure are directed to a sterile barrier system for medical device packaging. The sterile barrier system includes a plurality of hollow fibers filled with one or more agents. The agents can be self-healing agents and/or color indicator agents. The sterile barrier system can include one or more layers (e.g., a web of material) of woven fibers, nonwoven fibers, and/or fiber reinforced paper, wherein each of the layers can include a plurality of hollow fibers filled with one or more agents. The sterile barrier system can be breathable. For example, the sterile barrier system can include a first layer forming a breathable sterile barrier. The first layer can include a plurality of first fibers. Each of the first fibers can include a hollow fiber body and a first agent contained within the hollow fiber body. The fibers are configured such that the first agent flows from the corresponding hollow fiber body when the hollow fiber body is severed. With this construction, in the event the sterile barrier system is subjected to damage, the first agent is released from any hollow fiber body severed by the damage and effects one or both of repair or visual indication of a possible integrity breach. In some embodiments, the first agent is a self-healing agent or substance. In other embodiments, the first agent is a dye or other color indicator. In some embodiments, the first layer is an electrospun layer or web. In some embodiments, the first layer includes a plurality of second fibers, each with a hollow fiber body containing a second agent that differs from the first agent. For example, in some embodiments, the first agent can be a self-healing agent and the second agent can be a dye. In other embodiments, the first fibers can contain a first self-healing agent and the second fibers can contain a catalyst or initiator agent, wherein the self-healing agent reacts with the catalyst or initiator agent to form a substance that can heal or repair damage to the sterile barrier. In yet other embodiments, the sterile barrier system includes a second layer including a plurality of second fibers secured relative to the first layer including a plurality of first fibers. Each of the second fibers includes a hollow fiber body containing a second agent that differs from the first agent. For example, the first agent can be a dye having a first color, and the second agent can be a dye having a second color differing from the first color. With these and related embodiments, damage to the sterile barrier system that progresses only to the first layer will result in a visual indication to a user of the first color, whereas damage to the sterile barrier that progresses to both the first and second layers will result in a visual indication to a user of the second color. Thus, the exhibited color can provide a user with a visual indication of an extent of damage to the sterile barrier system. In some embodiments, two or more color agents can combine to form a color agent having a differing color from the two or more color agents.

### BRIEF DESCRIPTION OF DRAWINGS

FIG. 1 is a simplified, side cross-sectional view of a sterile barrier system in accordance with principles of the present disclosure;

FIG. 2A is an enlarged, simplified cross-sectional view of a fiber useful with the sterile barrier system of FIG. 1;

FIG. 2B illustrates the fiber of FIG. 2A in a severed condition;

FIG. 3A illustrates formation of a void into the sterile barrier system of FIG. 1;

FIG. 3B illustrates functioning of the sterile barrier system of FIG. 1 and incorporating a self-healing agent in response to the void;

FIG. 4A is a simplified side cross-sectional view of a layer useful with the barrier systems of the present disclosure;

FIGS. 4B and 4C illustrate functioning of the layer of FIG. 4A in response to the creation of a void therein;

FIG. 5A illustrates formation of a void into the sterile barrier system of FIG. 1;

FIG. 5B illustrates functioning of the sterile barrier system of FIG. 1 and incorporating a dye agent in response to the void;

FIG. 6A is a simplified side cross-sectional view of a layer useful with the barrier systems of the present disclosure;

FIG. 6B illustrates functioning of the layer of FIG. 6A in response to the creation of a void therein;

FIG. 7 is a simplified, side cross-sectional view of a sterile barrier system in accordance with principles of the present disclosure;

FIG. 8 is a simplified, side cross-sectional view of a sterile barrier system in accordance with principles of the present disclosure;

FIG. 9A is a simplified, side cross-sectional view of a sterile barrier system in accordance with principles of the present disclosure;

FIGS. 9B and 9C illustrate functioning of the sterile barrier system of FIG. 9A in response to the creation of a void therein;

FIG. 10 is a simplified, side cross-sectional view of a sterile barrier system in accordance with principles of the present disclosure;

FIG. 11A is a simplified, side cross-sectional view of a sterile barrier system in accordance with principles of the present disclosure;

FIG. 11B illustrates functioning of the sterile barrier system of FIG. 11A in response to the creation of a void therein;

FIG. 12 is a simplified cross-sectional view of a packaged good article in accordance with principles of the present disclosure; and

FIG. 13 is a simplified cross-sectional view of a packaged good article in accordance with principles of the present disclosure.

### DETAILED DESCRIPTION

Specific embodiments of the present disclosure are now described with reference to the figures, wherein like reference numbers indicate identical or functionally similar elements.

Aspects of the disclosure provide breathable sterile barrier systems that are beneficial for use with sterile packaging for medical items, such as the packaging of medical devices where a breathable sterile barrier is required (e.g., stents, prosthesis, surgical instruments, catheters, etc.). Breathable materials of the present disclosure are those whose microporosities permit air permeability in such a way as to allow changes in atmospheric pressure (e.g., 0.5-10 psi) without breaking their seals, and are suitable for sterilization (e.g., ethylene oxide sterilization). The breathable sterile barrier systems of the present disclosure are permeable to sterilizing gases such as vapor phase hydrogen peroxide, ozone, ethylene oxide, and steam, yet impermeable to potentially contaminating microorganisms.

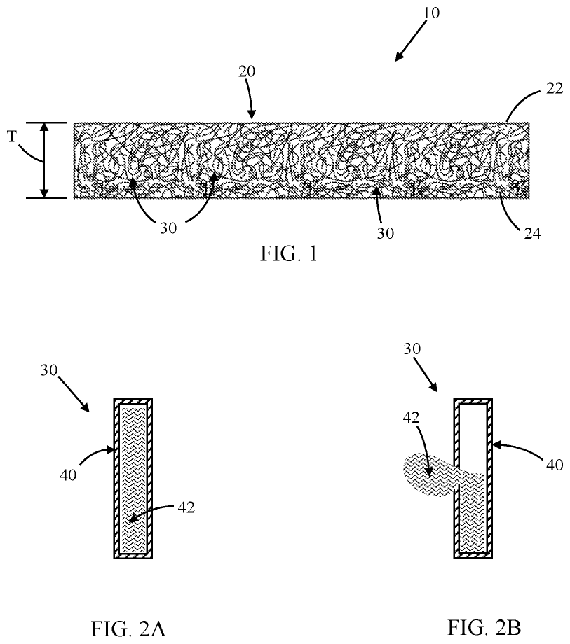

With the above in mind, one example of a sterile barrier system 10 of the present disclosure is schematically shown in FIG. 1 and includes a layer 20 forming a breathable sterile barrier. The layer 20 defines a first side 22 opposite a second side 24. A thickness T of the sterile barrier system 10 is defined by the layer 20 (i.e., distance between the first side 22 and second side 24) and is exaggerated in the illustration of FIG. 1 for ease of understanding. The layer 20 can be a web of material, such as a nonwoven web (i.e., a fabric that has a structure of individual fibers or filaments which are randomly and/or unidirectionally interlaid in a mat-like fashion). In some embodiments, the web of material can be an electrospun web. The layer 20 includes at least a plurality of first fibers 30 (several of which are referenced generally in FIG. 1). Each of the first fibers 30 can have a similar construction, an example of which is depicted schematically in FIG. 2A. It will be understood that the simplified representation of the first fiber 30 in FIG. 2A is for ease of explanation and is no way limiting of a particular size and/or shape of the first fiber 30. Moreover, the first fibers 30 as they exist in the layer 20 of FIG. 1 can each have a range of differing shapes, sizes (e.g., lengths) and orientations. A general construction of each of the first fibers 30 includes a hollow fiber body or shell 40 and an agent 42 contained within, or encapsulated by, the hollow fiber body 40. In at least the normal arrangement or configuration of FIG. 2A, the agent 42 is in a liquid or flowable state. With this construction, when the hollow fiber body 40 is severed, the agent 42 readily flows or wicks outwardly from the hollow fiber body 40 as in FIG. 2B for reasons made clear below.

Returning to FIG. 2A, the hollow fiber body 40 can be considered a micro-fiber, having an outer diameter (or maximum outer dimension with embodiments in which the hollow fiber body 40 has a shape other than circular in cross-section) on the order of 0.05 to 20 micrometers. A material of the hollow fiber body 40 can assume various forms, and is generally a polymer material, for example a biocompatible thermoplastic polymer. In some embodiments, a thermoplastic polymer material conducive to forming the hollow fiber body 40 via electrospinning is employed. In some embodiments, the hollow fiber body 40 can include a material such as polyethylene terephthalate, glycol, polyethylene, etc., or combinations thereof.

The agent 42 can assume a variety of forms and is generally a biocompatible solution. A formulation of the agent 42 is selected to exhibit one of a self-healing property or a colorant property.

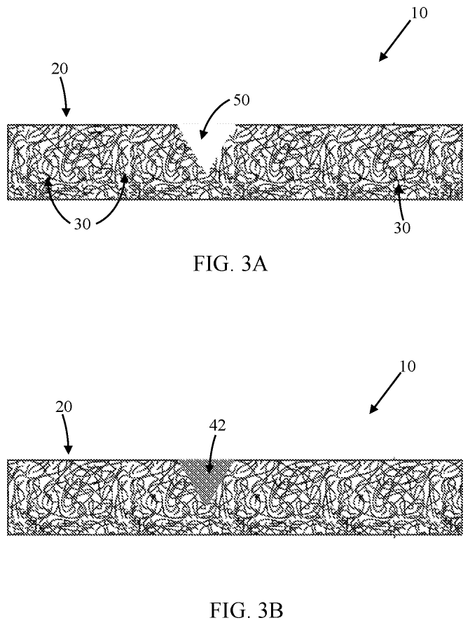

With embodiments in which the agent 42 is formulated as a self-healing substance or material, the agent 42 can be, or can include, a biocompatible adhesive. When released from the confines of the hollow fiber body 40, the so-formulated self-healing agent 42 flows onto surrounding surfaces and solidifies or cures, thus “healing” a tear or breach in the surrounding area. For example, FIG. 3A is a simplified representation of a void 50 (e.g., tear, puncture, etc.) imparted into the layer 20 of FIG. 1. The body or force acting on the layer 20 to create the void 50 will pass through and sever one or more (likely many) of the first fibers 30 (several of which are referenced generally) due to the random distribution of the first fibers 30 within the layer. Under these circumstances, the self-healing agent 42 will flow or wick from the so-severed first fiber(s) 30 into the void 50 and then cure. Thus, and as shown in FIG. 3B, the cured self-healing agent 42 partially or completely fills the void 50, effectively repairing the damage. With these and related embodiments, the repaired layer 20 can continue to serve as a breathable sterile barrier appropriate for medical item packaging.

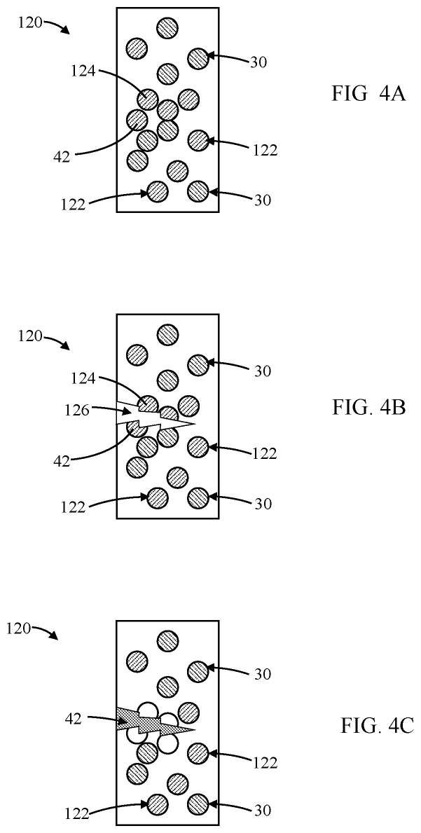

Returning to FIG. 2A, the self-healing material optionally employed with or as the agent 42 can be a biocompatible adhesive, an elastomer configured to “self-heal” through surface cohesion, etc. In other embodiments, the self-healing substance can be formulated to more readily cure and/or bond to surrounding surfaces in the presence of a catalyst or initiator agent. For example, FIG. 4A illustrates, in simplified form, portions of a layer 120 in accordance with principles of the present disclosure and useful as, or as part of, a sterile barrier system. The layer 120 includes the plurality of the first fibers 30 (several of which are labeled) as described above, along with a plurality of second fibers 122 (several of which are labeled). The first fibers 30 each include the hollow fiber body 40 (FIG. 2A) containing the agent 42 formatted with a self-healing substance. The second fibers 122 similarly include a hollow fiber body (not labeled in FIG. 4A, the same as or similar to the hollow fiber body 40 of FIG. 2A) containing a catalyst or initiator agent 124. A formulation of the initiator agent 124 is selected to promote curing and/or adhering of the self-healing agent 42 (e.g., the self-healing agent 42 is a liquid resin that hardens upon reacting with the initiator agent 124. When a void 126 is formed in the layer 120 (e.g., tear, puncture, etc.), the hollow fiber body of at least some of the first fibers 30 and of at least some of the second fibers 122 is severed as shown in FIG. 4B. The self-healing agent 42 of the so-severed first fibers 30 and the initiator agent 124 of the so-severed second fibers 122 flow or wick outwardly into the void 126 and react or combine with one another. The released self-healing agent 42 is thus caused to cure, harden and or/adhere to surrounding surfaces, partially or completely filling (and thus repairing “healing”) the void 126 as shown in FIG. 4C.

Returning to FIG. 2A, in other embodiments the agent 42 is or includes a biocompatible dye or other liquid colorant. The dye agent can assume a variety of forms, and in some embodiments has a color differing from a color of the hollow fiber body 40. In some embodiments, the hollow fiber body 40 is opaque or semi-opaque such that a color of the dye agent 42 is not readily visible when encapsulated within the hollow fiber body 40.

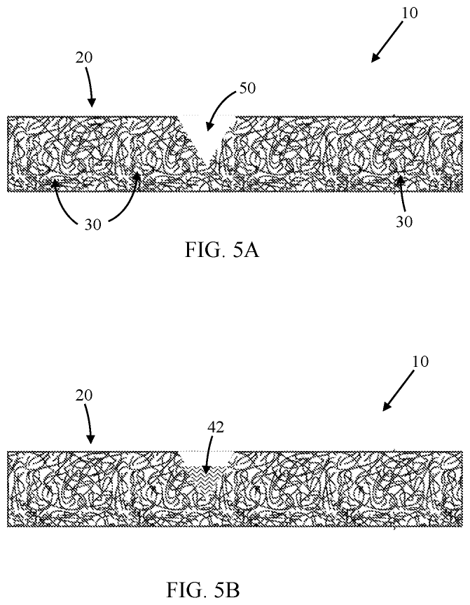

In some embodiments, a color of the dye agent 42 is selected to provide a visual “warning” of a possible breach or defect in the layer 20 (FIG. 1). For example, a color of the dye agent 42 can be red, although any other color is also acceptable. Regardless, when released from the confines of the hollow fiber body 40, the so-formulated dye agent 42 flows or wicks onto surrounding surfaces and is visually perceptible. For example, FIG. 5A is a simplified representation of the void 50 (e.g., tear, puncture, etc.) imparted into the layer 20 of FIG. 1. The body or force acting on the layer 20 to create the void 50 will pass through and sever one or more (likely many) of the first fibers 30 (several of which are referenced generally) due to the random distribution of the first fibers 30 within the layer. Under these circumstances, the dye agent 42 will flow or wick from the so-severed first fiber(s) 30 into the void 50 and become visually perceptible from an exterior of the layer 20 as generally reflected by FIG. 5B. The exposed dye agent 42 thus indicates or alerts a user that an integrity breach may have occurred.

Returning to FIGS. 1 and 2A, regardless of a composition of the agent 42, in some embodiments the hollow fiber body 40 of the first fibers 30 is formed by an electrospinning production process in which electric forces are employed to draw charged threads of polymer solutions or polymer melts up to fiber diameters on the order of hundreds of nanometers. The fibers are carried by a gas stream and are deposited on a collection surface to form a layer (e.g., web) of randomly disbursed fibers. The agent 42 can be dispensed or injected into the so-formed hollow fiber bodies 40 in various manners and at various stages of the production process. For example, the agent 42 can be dispensed as the corresponding hollow fiber body 40 is being formed, after formation of the hollow fiber body 40 but prior to formation of a web, and/or after the hollow fiber bodies 40 have been collected into a web. Regardless, once the hollow fiber body 40 has been partially or completely filled with a volume of the agent 42, the hollow fiber body 40 can be closed to encapsulate or contain the agent 42 in various manners. Known hollow fiber manufacturing techniques other than electrospinning can be employed to generate the first fibers 30 in other embodiments.

In some embodiments, the layer 20 is primarily (i.e., greater than 50 percent by weight) comprised of the first fibers 30, alternatively at least 60 percent by weight, alternatively at least 70 percent by weight, alternatively at least 80 percent by weight. In other embodiments, the layer 20 can include components in addition to the first fibers 30, for example components that further render the layer 20 to be a viable, breathable sterile barrier. For example, other fibers (that may or may not be hollow fibers) can be combined with the first fibers 30 to collectively establish a desired porosity across the thickness T of the sterile barrier system 10. The desired porosity may be permeable to sterilizing gases such as vapor phase hydrogen peroxide, ozone, ethylene oxide, and steam, yet impermeable to potentially contaminating microorganisms (e.g., a porosity on the order of 6 microns). Alternatively or in addition, components other than fibers that maintain or contribute to the desired porosity of the sterile barrier system 10 can be included with the layer 20.

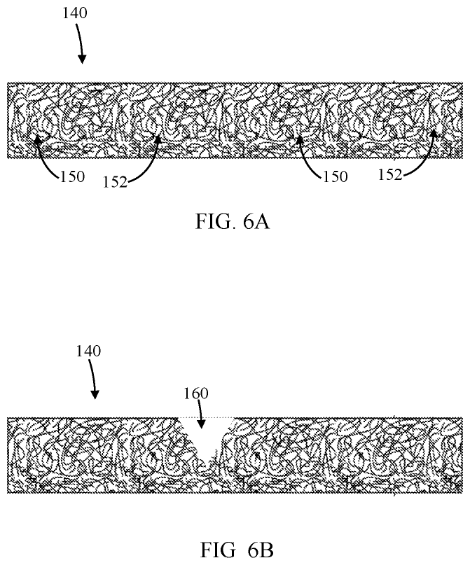

FIG. 6A illustrates, in simplified form, portions of another layer 140 in accordance with principles of the present disclosure and useful as, or as part of, a sterile barrier system. The layer 140 includes a plurality of first fibers 150 (several of which are labeled) and a plurality of second fibers 152 (several of which are labeled). The first fibers 150 each include a hollow fiber body containing an agent formatted as a self-healing substance as described above. The second fibers 152 each include a hollow fiber body containing an agent formatted as a dye as described above. With reference to FIG. 6B, when a void 160 is formed in the layer 140 (e.g., tear, puncture, etc.), the hollow fiber body of one or more of the first fibers 150 and of one or more of the second fibers 152 may be severed. Under these circumstances, the self-healing agent of the so-severed first fiber(s) 150 and the dye agent of the so-severed second fiber(s) 152 flow or wick outwardly into the void 160. The released self-healing agent serves to partially or completely repair the void 160, whereas the released dye agent is visually perceptible by a user and provides a visual indication of a possible integrity breach.

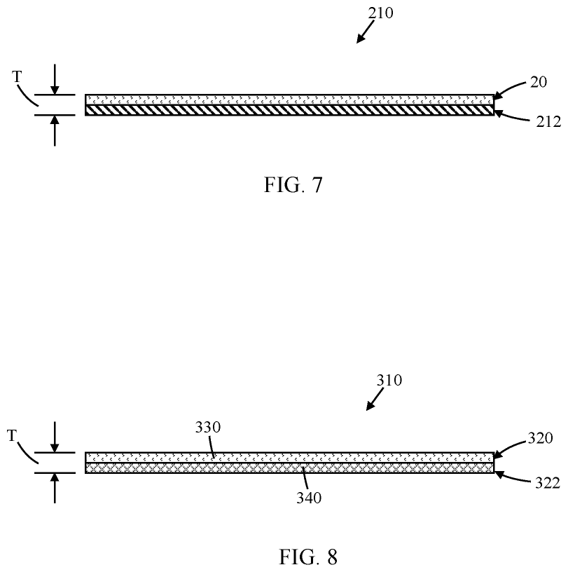

In other embodiments, layers (e.g., webs) of the present disclosure (e.g., the layer 20 of FIG. 1) can formed separately from, and secured to, one or more additional layers. For example, FIG. 7 illustrates another sterile barrier system 210 in accordance with principles of the present disclosure. The sterile barrier system 210 includes the layer 20 as described above, and at least one auxiliary layer 212. The auxiliary layer 212 can assume various forms (e.g., film, web, paper, etc.) compatible with the layer 20 and conducive, for example, in rendering the barrier system 210 to be a breathable, sterile barrier system. For example, in some embodiments, the layer 20 alone may not provide a desired barrier to bacteria and/or microorganism of interest (e.g., a porosity of the layer 20 may be insufficient to serve as a barrier to some bacteria and/or microorganisms). With these and related embodiments, the auxiliary layer 212 is configured to, alone or in combination with the layer 20, create the desired porosity across the thickness T of the sterile barrier system 210 (e.g., the auxiliary layer 212 can have a conventional sterile barrier construction such as Tyvek® material, medical-grade paper, etc.). The layer 20 can be secured to the auxiliary layer 212 in various manners complementary to their compositions (e.g., adhesive, bonding, welding, heat seal, etc.). Regardless, the layer 20 provides one or both of the self-healing or color indicator features as described above in the event of a possible integrity breach in the sterile barrier system 210.

FIG. 8 illustrates another sterile barrier system 310 in accordance with principles of the present disclosure. The sterile barrier system 310 includes a first layer 320 and a second layer 322. The first layer 320 can have one or more features that are the same as or similar to the layer 20 (FIG. 1) described above and includes a plurality of first fibers 330 (referenced generally). The second layer 322 can also have one or more features that are the same as or similar to the layer 20 described above and includes a plurality of second fibers 332 (referenced generally). The first fibers 330 each include a hollow fiber body containing an agent formatted as a self-healing substance as described above. The second fibers 332 each include a hollow fiber body containing an agent formatted as a dye as described above. The first and second layers 320, 322 can be secured to one another in various manners (e.g., adhesive, bond, weld, etc.). The first and second layers 320, 322 can combine to establish the sterile barrier system 310 as a breathable sterile barrier appropriate for sterilizing a contained medical item and maintaining the sterile environment of the contained medical item. For example, the first and second layers 320, 322 collectively establish a desired porosity across the thickness T of the sterile barrier system 310 that is permeable to sterilizing gases such as vapor phase hydrogen peroxide, ozone, ethylene oxide, and steam, yet impermeable to potentially contaminating microorganisms (e.g., a porosity on the order of 6 microns). Alternatively, one or more additional layers can be secured to one or both of the first and second layers 320, 322 to assist in creating the desired porosity across the thickness T of the sterile barrier system 310.

When a void is formed in the sterile barrier system 310 (e.g., tear, puncture, etc.), the hollow fiber body of one or more of the first fibers 330 and/or of one or more of the second fibers 332 may be severed. Under these circumstances, the self-healing agent of the so-severed first fiber(s) 330 and/or the dye agent of the so-severed second fiber(s) 332 will flow or wick outwardly into the void. The released self-healing agent serves to partially or completely repair the void, whereas the released dye agent is visually perceptible by a user and provides a visual indication of a possible integrity breach.

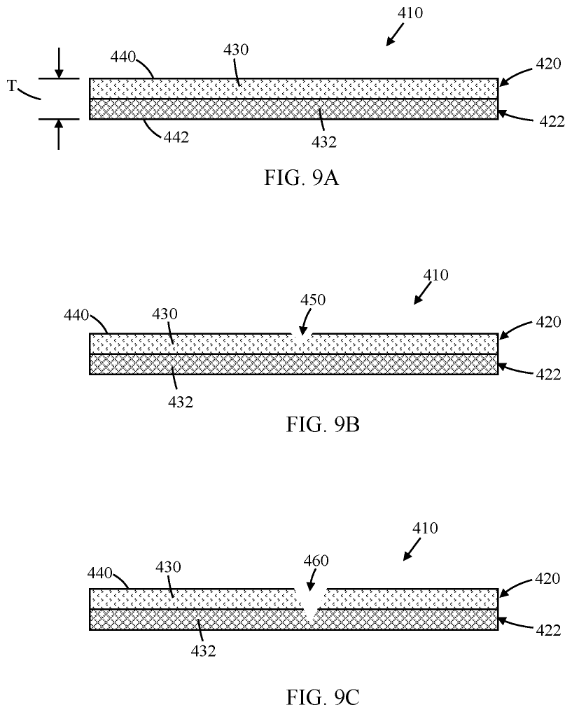

FIG. 9A illustrates another sterile barrier system 410 in accordance with principles of the present disclosure. The sterile barrier system 410 includes a first layer 420 and a second layer 422. The first layer 420 can have one or more features that are the same as or similar to the layer 20 (FIG. 1) described above and includes a plurality of first fibers 430 (referenced generally). The second layer 422 can also have one or more features that are the same as or similar to the layer 20 described above and includes a plurality of second fibers 432 (referenced generally). The first fibers 430 each include a hollow fiber body containing an agent formatted as a first dye as described above, with the first dye having a first color. The second fibers 432 each include a hollow fiber body containing an agent formatted as a second dye as described above, with the second dye having a second color that differs from the first color. The first and second layers 420, 422 can be secured to one another in various manners (e.g., adhesive, bond, weld, etc.). The first and second layers 420, 422 can combine to establish the sterile barrier system 410 as a breathable sterile barrier appropriate for sterilizing a contained medical item and maintaining the sterile environment of the contained medical item. For example, the first and second layers 420, 422 collectively establish a desired porosity across the thickness T of the sterile barrier system 410 that is permeable to sterilizing gases such as vapor phase hydrogen peroxide, ozone, ethylene oxide, and steam, yet impermeable to potentially contaminating microorganisms (e.g., a porosity on the order of 6 microns)). Alternatively, one or more additional layers can be secured to one or both of the first and second layers 420, 422 to assist in creating the desired porosity across the thickness T of the sterile barrier system 410.

Upon final construction, a surface of the first layer 420 opposite the second layer 422 defines an exterior side 440 of the sterile barrier system 410, and a surface of the second layer 422 opposite the first layer 420 defines an interior side 442 of the sterile barrier system 410. The terms “exterior” and “interior” are relative to an intended orientation of the sterile barrier system 410 relative to a medical item when the sterile barrier system 410 is employed with, or as, a medical item packaging; the interior side 442 is the side closest to the medical item and the exterior side 440 is the side further away from the medical item (and thus is the exterior side of the packaging). With these conventions in mind, the first color (of the first dye provided with the first fibers 430) and the second color (of the second dye provided with the second fibers 432) can be selected to intuitively indicate a depth of penetration of a void into the sterile barrier system 410. For example, the first color can be green (or some other color intuitively indicative of lesser damage) and the second color can be red (or some other color intuitively indicative of greater damage).

With reference to FIG. 9B, when a void 450 is formed into and through the exterior side 440 of the sterile barrier system 410 that does not otherwise extend to the second layer 422, none of the second fibers 432 will be severed. Instead, only one or more of the first fibers 430 will be severed, with the first dye of the so-severed first fiber(s) 430 flowing or wicking outwardly into the void 450 and becoming visible from the exterior side 440. Where the first color (of the first dye) is intuitively perceived as representing minor or lesser damage (e.g., green), a viewer is readily apprised that some minor damage may have occurred.

With reference to FIG. 9C, however, when a void 460 is formed into and through the exterior side 440 of the sterile barrier system 410 that does extend to the second layer 422, one or more of the second fibers 432 will be severed. The second dye of the so-severed second fiber(s) 432 flows or wicks outwardly into the void 460 (as does the first dye of any severed first fibers 430) and becomes visible from the exterior side 440. Where the second color (of the second dye) is intuitively perceived as representing substantive or greater damage (e.g., red), a viewer is readily apprised that substantive damage may have occurred. In some embodiments, visual presence of only the first dye may indicate to a user that, despite having sustained damage, the efficacy of the sterile barrier system 410 has not been compromised and the medical device stored within the sterile barrier system 410 remains uncontaminated, safe, and otherwise usable. Alternatively, visual presence of the second dye, either alone or in combination with visual presence of the first dye, may indicate to a user that the sterile barrier system 410 has sustained damage that compromises its efficacy and the medical device stored within the sterile barrier system 410 may be contaminated, unsafe, or otherwise unusable.

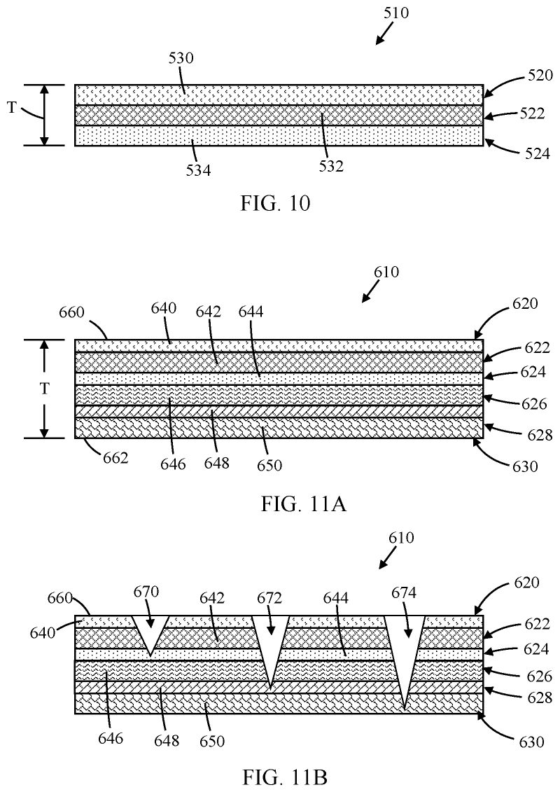

FIG. 10 illustrates another sterile barrier system 510 in accordance with principles of the present disclosure. The sterile barrier system 510 includes a first layer 520, a second layer 522, and a third layer 524. The first layer 520 can have one or more features that are the same as or similar to the layer 20 (FIG. 1) described above and includes a plurality of first fibers 530 (referenced generally). The second layer 522 can also have one or more features that are the same as or similar to the layer 20 described above and includes a plurality of second fibers 532 (referenced generally). The third layer 524 can also have one or more features that are the same as or similar to the layer 20 described above and includes a plurality of third fibers 534 (referenced generally). The first fibers 530 each include a hollow fiber body containing a first agent as described above; the second fibers 532 each include a hollow fiber body containing a second agent as described above and differing from the first agent; the third fibers 534 each include a hollow fiber body containing a third agent as described above and differing from at least one of the first and second agents. In some embodiments, at least one of the first, second, and third agents is formulated as a self-healing substance, and at least one of the first, second, and third agents is formulated as a dye or other color indicator. For example, the first agent (of the first fibers 530) can be a dye having a first color, the second agent (of the second fibers 532) can be a self-healing substance, and the third agent (of the third fibers 534) can be a dye having a second color differing from the first color (e.g., the first color of the first agent can be green (or some other color intuitively indicative of lesser damage) and the second color of the third agent can be red (or some other color intuitively indicative of greater damage) as described above with respect to the sterile barrier system 410 (FIG. 9A)). In other embodiments, the first agent can be a dye having a first color, the second agent can be a dye having a second color, and the third agent can be a dye having a third color differing from at least one of the first color and the second color.

The first, second, and third layers 520, 522, 524 can be secured to one another in various manners (e.g., adhesive, bond, weld, etc.). The first, second, and third layers 520, 522, 524 can combine to establish the sterile barrier system 510 as a breathable sterile barrier appropriate for sterilizing a contained medical item and maintaining the sterile environment of the contained medical item. For example, the first, second, and third layers 520, 522, 524 collectively establish a desired porosity across the thickness T of the sterile barrier system 510 that is permeable to sterilizing gases such as vapor phase hydrogen peroxide, ozone, ethylene oxide, and steam, yet impermeable to potentially contaminating microorganisms (e.g., a porosity on the order of 6 microns)). Alternatively, one or more additional layers can be secured to one or more of the first, second, and third layers 520, 522, 524 to assist in creating the desired porosity across the thickness T of the sterile barrier system 510.

FIG. 11A illustrates another sterile barrier system 610 in accordance with principles of the present disclosure. The sterile barrier system 610 includes a first layer 620, a second layer 622, a third layer 624, a fourth layer 626, a fifth layer 628, and a sixth layer 630 arranged relative to one another in the order shown. Each of the first, second, third, fourth, fifth, and sixth layers 620, 622, 624, 626, 628, 630 can have features the same as or similar to the layer 20 (FIG. 1) described above. The first layer 620 includes a plurality of first fibers 640 (referenced generally); the second layer 622 includes a plurality of second fibers 642 (referenced generally); the third layer 624 includes a plurality of third fibers 644 (referenced generally); the fourth layer 626 includes a plurality of fourth fibers 646; the fifth layer 628 includes a plurality of fifth fibers 648 (referenced generally); and the sixth layer 630 includes a plurality of sixth fibers 650. The first, second, third, fourth, fifth, and sixth fibers 640, 642, 644, 646, 648, 650 each include a hollow fiber body containing an agent as described above, such as first, second, third, fourth, fifth, and sixth agents, respectively. In some embodiments, the first, third, and fifth agents (of the first, third and fifth fibers 640, 644, 648, respectively) are formulated as a self-healing substance, and the second, fourth, and sixth agents (of the second, fourth, and sixth fibers 642, 646, 650, respectively) are formulated as a dye or other color indicator. For example, the second agent (of the second fibers 642) can be a dye having a first color, the fourth agent (of the fourth fibers 646) can be a dye having a second color differing from the first color, and the sixth agent (of the sixth fibers 650) can be a dye having a third color differing from at least one of the first color and the second color.

The first, second, third, fourth, fifth, and sixth layers 620, 622, 624, 626, 628, 630 can be secured to one another in various manners (e.g., adhesive, bond, weld, etc.). The first, second, third, fourth, fifth, and sixth layers 620, 622, 624, 626, 628, 630 can combine to establish the sterile barrier system 610 as a breathable sterile barrier appropriate for sterilizing a contained medical item and maintaining the sterile environment of the contained medical item. For example, the first, second, third, fourth, fifth, and sixth layers 620, 622, 624, 626, 628, 630 collectively establish a desired porosity across the thickness T of the sterile barrier system 610 that is permeable to sterilizing gases such as vapor phase hydrogen peroxide, ozone, ethylene oxide, and steam, yet impermeable to potentially contaminating microorganisms (e.g., a porosity on the order of 6 microns). Alternatively, one or more additional layers can be secured to one or more of the first, second, third, fourth, fifth, and sixth layers 620, 622, 624, 626, 628, 630 that assist in creating the desired porosity across the thickness T of the sterile barrier system 610.

Upon final construction, a surface of the first layer 620 opposite the remaining layers 622, 624, 626, 628, 630 defines an exterior side 660 of the sterile barrier system 610, and a surface of the sixth layer 630 opposite the remaining layers 620, 622, 624, 666, 628 defines an interior side 662 of the sterile barrier system 610. The terms “exterior” and “interior” are relative to an intended orientation of the sterile barrier system 610 relative to a medical item when the sterile barrier system 610 is employed with, or as, medical item packaging. The interior side 662 is the side closest to the medical item and the exterior side 660 is the side further away from the medical item (and thus is the exterior side of the packaging). Thus, the second layer 622 is more proximate the exterior side 660 as compared to the fourth and sixth layers 626, 630; the sixth layer 630 is more proximate the interior side 662 as compared to the second and fourth layers 622, 626; and the fourth layer 626 is approximately mid-way between the exterior and interior sides 660, 662. With these conventions in mind, the first, second, and third colors can be selected to intuitively indicate a depth of penetration of a void into the sterile barrier system 610. For example, the first color of the dye agent of the second fibers 642 can be green or some other color intuitively indicative of lesser damage; the second color of the dye agent of the fourth fibers 646 can be yellow or some other color intuitively indicative of caution or intermediate damage; and the third color of the dye agent of the sixth fibers 650 can be red or some other color intuitively indicative of greater severe damage. As noted above, in some embodiments, visual presence of only the first dye, or the first dye and the second dye, may indicate to a user that, despite having sustained damage, the efficacy of the sterile barrier system 610 has not been compromised and the medical device stored within the sterile barrier system 610 remains uncontaminated, safe, and otherwise usable. Alternatively, visual presence of the third dye, either alone or in combination with visual presence of one or both of the first dye and the second dye, may indicate to a user that the sterile barrier system 610 has sustained damage that compromises its efficacy and the medical device stored within the sterile barrier system 610 may be contaminated, unsafe, or otherwise unusable.

With reference to FIG. 11B, when a void 670 is formed into and through the exterior side 660 of the sterile barrier system 610 that progresses to, but not beyond, the third layer 624, one or more of the first fibers 640 and/or one or more of the third fibers 644 may be severed, with the released self-healing agent flowing or wicking into the void 670 to partially or completely repair the void 670. Further, one or more of the second fibers 642 may be severed, with the first dye (having the first color) flowing or wicking outwardly into the void 670 and becoming visible from the exterior side 660. None of the fourth or sixth fibers 646, 650 are severed meaning only the first dye/first color is visible from the exterior side 660 at the void 670. Where the first color (of the first dye) is intuitively perceived as representing minor or lesser damage (e.g., green), a viewer is readily apprised that some minor damage may have occurred.

When a void 672 is formed into and through the exterior side 660 of the sterile barrier system 610 that progresses to, but not beyond, the fifth layer 628, one or more of the first fibers 640, one or more of the third fibers 644, and/or one or more of the fifth fibers 648 may be severed, with the released self-healing agent flowing or wicking into the void 672 to partially or completely repair the void 672. Further, one or more of the fourth fibers 646 may be severed, with the second dye (having the second color) flowing or wicking outwardly into the void 672 and becoming visible from the exterior side 660 (as will the first dye from any severed ones of the second fibers 642). None of the sixth fibers 650 are severed meaning the second dye/second color is visible at the void 672, but the third dye/third color is not. Where the second color (of the second dye) is intuitively perceived as representing caution or intermediate damage (e.g., yellow), a viewer is readily apprised that some potentially substantive damage may have occurred.

When a void 674 is formed into and through the exterior side 660 of the sterile barrier system 610 that progresses to the sixth layer 630, one or more of the first fibers 640, one or more of the third fibers 644, and/or one or more of the fifth fibers 648 may be severed, with the released self-healing agent flowing or wicking into the void 674 to partially or completely repair the void 674. Further, one or more of the sixth fibers 650 may be severed, with the third dye (having the third color) flowing or wicking outwardly into the void 674 and becoming visible from the exterior side 660 (as will the first dye from any severed ones of the second fibers 642 and the second dye from any severed ones of the fourth fibers 646). Where the third color (of the third dye) is intuitively perceived as representing substantive or greater damage (e.g., red), a viewer is readily apprised that substantive or deep damage may have occurred.

The green, yellow, and red color scheme as described above for the sterile barrier system 610 is intuitively understood as similar to a traffic light, where the color red is an immediate warning. Alternatively, other color schemes may be employed.

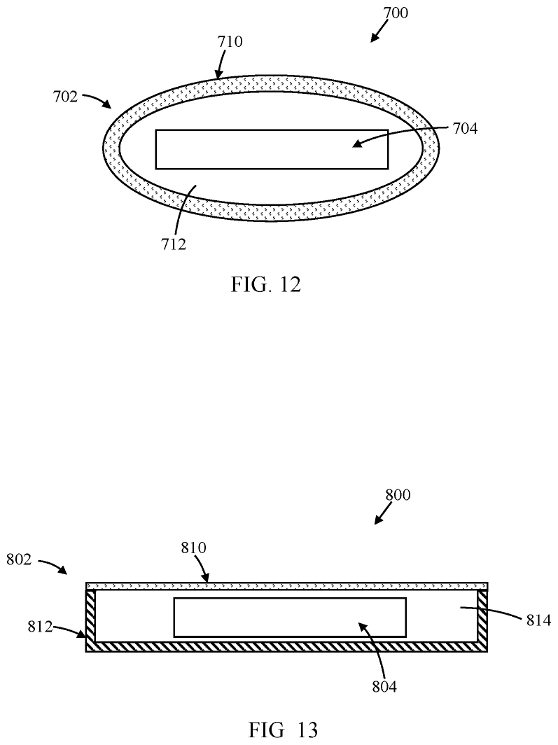

The breathable sterile barrier systems of the present disclosure can be utilized with or for the packaging of medical item(s) or other items in various manners. For example, FIG. 12 illustrates, in simplified form, one example of a packaged good article 700 of the present disclosure that includes packaging 702 containing at least one medical item 704. The packaging 702 includes a sterile barrier system 710 formed into or formatted as a sealed bag or pouch having an interior region 712 within which the medical item 704 is contained. The sterile barrier system 710 can be any of the sterile barrier systems as described herein (e.g., the sterile barrier system 10, 110, 210, 310, 410, 510, or 610) as described above. Regardless, the packaged good article 700 can be subjected to conventional sterilization processes, with the sterilizing gas passing through the sterile barrier system 710 to sterilize the medical item. Subsequently, the sterile barrier system 710 maintains the sterility of the medical item 704, preventing passage of potentially contaminating microorganisms from the external environment to the interior region 712. Further, the sterile barrier system 710 provides one or both of the self-healing or color indicator features as described above in the event of a possible integrity breach or damage.

FIG. 13 illustrates, in simplified form, another example of a packaged good article 800 of the present disclosure that includes packaging 802 containing at least one medical item 804. The packaging 802 includes a sterile barrier system 810 and a tray 812. The tray 812 is formed of a plastic material or the like and defines or forms an interior region 814 sized or shaped to receive the medical item 804. The sterile barrier system 810 is assembled across an open end of the tray 812 to enclose the medical item 804 within the interior region 814. The sterile barrier system 810 can be any of the sterile barrier systems as described herein (e.g., the sterile barrier system 10, 110, 210, 310, 410, 510, or 610) as described above. Regardless, the packaged good article 800 can be subjected to conventional sterilization processes, with the sterilizing gas passing through the sterile barrier system 810 to sterilize the medical item. Subsequently, the sterile barrier system 806 maintains the sterility of the medical item 804, preventing passage of potentially contaminating microorganisms from the external environment to the interior region 814. Further, the sterile barrier system 810 provides one or both of the self-healing or color indicator features as described above in the event of a possible integrity breach or damage.

Although the present disclosure has been described with reference to preferred embodiments, persons skilled in the art will recognize that changes can be made in form and detail without departing from the spirit and scope of the present disclosure.

What is claimed is:

1. A sterile barrier system for a medical device packaging, the system comprising:
a first layer forming a breathable sterile barrier, the first layer including a plurality of first fibers, each of the fibers of the plurality of first fibers including:
a severable hollow fiber body, and
a first flowable agent contained within the severable hollow fiber body.

2. The sterile barrier system of claim 1, wherein the first flowable agent is a self-healing agent.

3. The sterile barrier system of claim 2, wherein the self-healing agent is configured to flow from the corresponding severable hollow fiber body when the corresponding severable hollow fiber body is severed and repair a void in the first layer.

4. The sterile barrier system of claim 1, wherein the first flowable agent is a dye.

5. The sterile barrier system of claim 4, wherein a color of the dye is different from a color of the corresponding severable hollow fiber body in which the dye is contained.

6. The sterile barrier system of claim 5, wherein the color of the dye is not visually perceptible when the dye is contained within the corresponding severable hollow fiber body.

7. The sterile barrier system of claim 1, wherein the first layer is an electrospun web.

8. The sterile barrier system of claim 1, wherein the first layer further comprises a plurality of second fibers each including a severable hollow fiber body and a second flowable agent contained within the corresponding severable hollow fiber body.

9. The sterile barrier system of claim 8, wherein the first flowable agent is a self-healing agent and the second flowable agent is a dye.

10. A sterile barrier system for a medical device packaging, the system comprising:
a first layer including a first severable hollow fiber body and a first flowable agent contained within the first severable hollow fiber body;
a second layer secured relative to the first layer, the second layer including a second severable hollow fiber body and a second flowable agent contained within the second severable hollow fiber body.

11. The sterile barrier system of claim 10, wherein a property of the first flowable agent differs from a property of the second flowable agent.

12. The sterile barrier system of claim 11, wherein the first flowable agent is a self-healing agent and the second flowable agent is a dye.

13. The sterile barrier system of claim 11, wherein the first flowable agent is a dye having a first color and the second flowable agent is a dye having a second color differing from the first color.

14. The sterile barrier system of claim 10, further comprising:
a third layer secured relative to the first layer and the second layer, the third layer including
a third severable hollow fiber body and a third flowable agent contained within the third severable hollow fiber body.

15. The sterile barrier system of claim 14, wherein the first flowable agent is a dye having a first color, the second flowable agent is a dye having a second color differing from the first color, and the third flowable agent is a self-healing agent.

16. The sterile barrier system of claim 15, wherein the third layer is disposed between the first layer and the second layer.

17. The sterile barrier system of claim 14, wherein the first flowable agent is a dye having a first color, the second flowable agent is a dye having a second color differing from the first color, and the third flowable agent is a dye having a third color differing from at least one of the first color and the second color.

18. A medical device packaged in a sterile barrier system, the sterile barrier system comprising:
a plurality of fibers constructed as an electrospun web, each of the fibers of the plurality of fibers including:
a severable hollow fiber body, and
a flowable agent contained within the severable hollow fiber body.

19. The medical device packaged in a sterile barrier system of claim 18, wherein the flowable agent is a self-healing agent.

20. The medical device packaged in a sterile barrier system of claim 18, wherein the flowable agent is a dye having a color that is not visually perceptible when the dye is contained within the severable hollow fiber body.
